(19) United States
(12) Patent Application Publication
(10) Pub. No.: US 2015/0133326 A1
(43) Pub. Date: May 14, 2015

(54) BIOMARKERS FOR RECOVERED HEART FUNCTION

(71) Applicant: The University of British Columbia, Vancouver (CA)

(72) Inventors: Bruce McManus, Vancouver (CA); Zsuzsanna Hollander, Vancouver (CA); Andrew Ignaszewski, West Vancouver (CA); George Schreiner, Los Altos Hills, CA (US); Janet E. Wilson-McManus, Vancouver (CA); Raymond T. Ng, Vancouver (CA); Robert Balshaw, Vancouver (CA); Paul Keown, Delta (CA); Robert McMaster, Vancouver (CA); Scott J. Tebbutt, Vancouver (CA)

(21) Appl. No.: 14/395,392

(22) PCT Filed: Apr. 18, 2013
(86) PCT No.: PCT/CA2013/000385

§ 371 Date: Oct. 17, 2014

Related U.S. Application Data
(60) Provisional application No. 61/635,173, filed on Apr. 18, 2012.

(51) Int. Cl. C12Q 1/68

(52) U.S. Cl. CPC C12Q 1/6883; C12Q 2600/112
USPC 506/9; 435/6.12; 435/7.92; 436/501

(57) ABSTRACT

Disclosed herein is a method for determining recovered heart function in a subject based in the biomarker ceruloplasmin in patient samples. Also disclosed are computer systems, kits and software.

### CROSS REFERENCE TO RELATED APPLICATIONS

This application claims priority to U.S. Provisional Patent Application No. 61/635,173, filed Apr. 18, 2012, incorporated by reference herein in its entirety.

### FIELD

This application is directed to the area of cardiology. The teachings relate to biomarkers for determining recovered heart function.

### BACKGROUND

Heart failure (HF) represents a disease with high mortality and morbidity rates. Due to the high prevalence, HF causes a big overall economic and social burden. In the United States alone, there were 5.8 million HF patients in 2006, and the estimated direct and indirect costs related to HF in the United States were $39.2 billion in 2010. Although the incidence of HF in the last decade has not decreased, survival rates have improved. As the population grows older and heart disease fatality rates decrease due to better treatment methods, the number of patients with HF continues to increase. In addition, HF treatment has also improved during the past three decades, leading to a growing number of patients with recovered heart function. However, under current clinical practice, these patients continue to be followed by heart failure specialists or cardiologists and their treatment is ongoing. This is due to a lack of guidelines for determining whether a patient has been “cured” of heart failure, as no current test exists for assessing which patients have recovered heart function and therefore require less medication and follow-up.

Thus, tests for assessing recovered heart function are needed. The methods and compositions of the present invention help to satisfy these and other needs for such tests.

### SUMMARY

Disclosed herein are compositions and methods for determining recovered heart function in a subject using biomarkers from a sample derived from the subject.

In a first aspect, the present invention provides a method for determining recovered heart function in a subject by obtaining a dataset associated with a sample obtained from the subject, wherein the dataset comprises at least one marker selected from Table 1; analyzing the dataset to determine data for the markers, where the data is positively correlated or negatively correlated with recovered heart function in the subject.

In an embodiment of this aspect, the dataset comprises data for at least two, three, four, five, six, seven, eight, nine, ten, eleven, twelve, thirteen, fourteen, fifteen, sixteen, seventeen, eighteen, nineteen, twenty or more markers; and further comprises analyzing the dataset to determine the expression level of the at least two, three, four, five, six, seven, eight, nine, ten, eleven, twelve, thirteen, fourteen, fifteen, sixteen, seventeen, eighteen, nineteen, twenty or more markers. In related embodiments, the method further comprises determining recovered heart function in the subject according to the relative number of positively correlated and negatively correlated marker expression level data present in the dataset.

In a second aspect, the present invention provides a method for determining recovered heart function in a subject by obtaining a first dataset associated with a first sample obtained from the subject before treatment, wherein the first dataset comprises at least one marker selected from Table 1; obtaining a second dataset associated with a second sample obtained from the subject after treatment, wherein the second dataset comprises at least one marker selected from Table 1; analyzing the first and second datasets to determine data for the markers, where the data is positively correlated or negatively correlated with recovered heart function in the subject.

In an embodiment of this aspect, the first dataset comprises data for at least two, three, four, five, six, seven, eight, nine, ten, eleven, twelve, thirteen, fourteen, fifteen, sixteen, seventeen, eighteen, nineteen, twenty or more markers selected from Table 1, and where the second dataset comprises data for at least two, three, four, five, six, seven, eight, nine, ten, eleven, twelve, thirteen, fourteen, fifteen, sixteen, seventeen, eighteen, nineteen, twenty or more markers selected from Table 1; and further comprises analyzing the first and second datasets to determine the expression level of the at least two, three, four, five, six, seven, eight, nine, ten, eleven, twelve, thirteen, fourteen, fifteen, sixteen, seventeen, eighteen, nineteen, twenty or more markers selected from Table 1. In related embodiments, the method further comprises determining recovered heart function in the subject according to the relative number of positively correlated and negatively correlated marker expression level data present in the first and second datasets.

In embodiments of the first and second aspects above, the sample obtained from the subject is a blood sample.

In other embodiments of the first and second aspects above, the data is nucleic acid expression data, which can be obtained using a nucleic acid microarray or PCR, for example, RT qPCR.

In other embodiments of the first and second aspects above, the data is protein expression data, which can be obtained using an antibody, such as an antibody which is labeled. In additional aspects, the protein expression data is obtained using mass spectrometry.

In other embodiments of the first and second aspects above, the method is implemented using one or more computers.

In further embodiments of the first and second aspects above, the first and/or second dataset is obtained stored on a storage memory.

In yet further embodiments of the first and second aspects above, obtaining the first and/or second dataset comprises receiving the first and/or second dataset directly or indirectly from a third party that has processed the sample to experimentally determine the first and/or second dataset.

In additional embodiments of the first and second aspects above, the subject is a human subject.

In additional embodiments of the first and second aspects above, the method further comprises assessing a clinical variable; and combining the assessment with the analysis of the first and/or second dataset to determine recovered heart function in a subject. In some embodiments, clinical variables can include left ventricular ejection fraction (LVEF) and New York Heart Association (NYHA) class.

In a third aspect, the present invention provides a method for determining recovered heart function in a subject by obtaining a sample from the subject, wherein the sample comprises at least one marker selected from Table 1; contacting the sample with a reagent; generating a complex between the reagent and the markers; detecting the complex to obtain a dataset associated with the sample, wherein the dataset comprises expression level data for the markers; and analyzing the expression level data for the markers, wherein the expression level of the markers is positively correlated or negatively correlated with recovered heart function in the subject.

In a fourth aspect, the present invention provides a method for determining recovered heart function in a subject by obtaining a first sample from the subject before treatment, wherein the first sample comprises at least one marker selected from Table 1; obtaining a second sample from the subject after treatment, wherein the second sample comprises at least one marker selected from Table 1; contacting the first and second samples with a reagent; generating a complex between the reagent and the markers; detecting the complex to obtain a dataset associated with the samples, wherein the dataset comprises expression level data for the markers; and analyzing the expression level data for the markers, where the expression level of the markers is positively correlated or negatively correlated with recovered heart function in the subject.

In a fifth aspect, the present invention provides a computer-implemented method for determining recovered heart function in a subject, by storing, in a storage memory, a dataset associated with a sample obtained from the subject, where the dataset comprises data for at least one marker selected from Table 1; and analyzing, by a computer processor, the dataset to determine the expression levels of the markers, where the expression levels are positively correlated or negatively correlated with recovered heart function in a subject.

In a sixth aspect, the present invention provides a computer-implemented method for determining recovered heart function in a subject, by storing, in a storage memory, a first dataset associated with a first sample obtained from the subject before treatment, wherein the first dataset comprises data for at least one marker selected from Table 1; storing, in a storage memory, a second dataset associated with a second sample obtained from the subject after treatment, wherein the second dataset comprises data for at least one marker selected from Table 1; and analyzing, by a computer processor, the first and second datasets to determine the expression levels of the markers, where the expression levels are positively correlated or negatively correlated with recovered heart function in the subject.

In a seventh aspect, the present invention provides a system for determining recovered heart function in a subject, the system including a storage memory for storing a dataset associated with a sample obtained from the subject, where the dataset comprises data for at least one marker selected from Table 1; and a processor communicatively coupled to the storage memory for analyzing the dataset to determine the expression levels of the markers, where the expression levels are positively correlated or negatively correlated with recovered heart function in the subject.

In an eighth aspect, the present invention provides a system for determining recovered heart function in a subject, the system including a storage memory for storing a first dataset associated with a first sample obtained from the subject before treatment, where the first dataset comprises data for at least one marker selected from Table 1; a storage memory for storing a second dataset associated with a second sample obtained from the subject after treatment, where the second dataset comprises data for at least one marker selected from Table 1; and a processor communicatively coupled to the storage memory for analyzing the first and second datasets to determine the expression levels of the markers, where the expression levels are positively correlated or negatively correlated with recovered heart function in the subject.

In an ninth aspect, the present invention provides a computer-readable storage medium storing computer-executable program code, the program code including program code for storing a dataset associated with a sample obtained from a subject, where the first dataset comprises data for at least one marker selected from Table 1; and program code for analyzing the dataset to determine the expression levels of the markers, where the expression levels of the markers are positively correlated or negatively correlated with recovered heart function in a subject.

In a tenth aspect, the present invention provides computer-readable storage medium storing computer-executable program code, the program code including program code for storing a first dataset associated with a first sample obtained from a subject before treatment, where the first dataset comprises data for at least one marker selected from Table 1; program code for storing a second dataset associated with a second sample obtained from a subject after treatment, where the second dataset comprises data for at least one marker selected from Table 1; and program code for analyzing the datasets to determine the expression levels of the markers, where the expression levels of the markers are positively correlated or negatively correlated with recovered heart function in a subject.

In an eleventh aspect, the present invention provides a kit for use in determining recovered heart function in a subject including a set of reagents comprising a plurality of reagents for determining from a sample obtained from the subject data for at least one marker selected from Table 1; and instructions for using the plurality of reagents to determine data from the samples. In some embodiments of this aspect, the instructions comprise instructions for conducting a protein-based assay.

In an twelfth aspect, the present invention provides a kit for use in determining recovered heart function in a subject, including a set of reagents consisting essentially of a plurality of reagents for determining from samples obtained from the subject data for at least one marker selected from Table 1; and instructions for using the plurality of reagents to obtain expression level data from the samples. In some embodiments of this aspect, the instructions comprise instructions for conducting a protein-based assay.

In a twelfth aspect, the present invention provides a use of a dataset associated with a sample obtained from a subject, wherein the dataset comprises at least one marker selected from Table 1; and wherein the dataset is analyzed to determine data for the markers and wherein the data is positively correlated or negatively correlated with recovered heart function in the subject.

In various embodiments of the above, the treatment for heart failure can be administration of a beta-blocker or ACE inhibitor.

In various embodiments of the above aspects, the at least one marker selected from Table 1 can be: Ceruloplasmin (ferroxidase) (CP); Alpha-2-antiplasmin (SERPINF2); Prothrombin (F2); Proteoglycan 4 (PRG4); Inter-alpha-trypsin inhibitor heavy chain H2 (ITIH2); Vitamin K-dependent protein (SPROS1); complement factor D(CFD); Coagulation factor (XF10); Vitamin K-dependent protein C (PROC); Apolipoprotein A-1 (APOA1); Clusterin (CLU); C4b-binding protein alpha chain (C4BPA); Vitronectin (VTN); Antithrombin-III (SERPINC1); coagulation factor IX (F9); insulin-like growth factor binding protein, acid labile subunit (IGFALS); angiotensinogen (AGT); and Serum amyloid P-component (APCS).

### BRIEF DESCRIPTION OF THE DRAWINGS

FIG. 1 shows an overview of biomarker discovery and validation analyses.

FIG. 2 shows results obtained in step 1 and 2 of the biomarker discovery analysis.

FIG. 3 shows the performance of the RHF biomarkers in the validation cohort.

### DETAILED DESCRIPTION

The research on biomarkers in HF is a progressively developing field in cardiology. Many novel biomarkers are currently under investigation, with some being identified as 1) indicators of myocardial injury, such as troponin, C-reactive protein; 2) active players in the myocardial remodeling process, such as galectin-3, matrix metalloproteinases (MMPs) and tissue inhibitors of metalloproteinases (TIMPs); or 3) involved in the neurohormonal activation in HF, such as B-type natriuretic peptide (BNP), adrenomedullin and Na. Several studies have attempted to identify biomarkers with a prognostic value in patients with new HF onset or after ventricular assist device (VAD) implantation. Others studies have investigated biomarkers that would “carry” information about HF recovery. The results of these studies are promising; nevertheless, more research is required before biomarker tests can be implemented in clinical practice. For a biomarker test to contribute significantly in clinical decision making and label a HF patient as recovered represents a novel, difficult and complex approach. However, such tests would address a current clinical unmet need, by decreasing the various medication-related side effects, improving HF management and patient quality of life, and lowering overall costs of HF treatment.

Given these considerations, the overall goal of the work disclosed herein was to identify novel blood biomarkers of recovered heart function in order to aid heart failure specialists in managing their patients. Heart transplantation is an excellent model for studying biomarkers of recovered heart function since before transplantation patients have heart failure and after receiving a new heart they are cured of the heart failure due to the new heart they received. The objectives of this work were therefore to 1) discover blood biomarkers using heart transplant data from the Biomarkers in Transplantation (BiT) initiative and 2) test these biomarkers in patients who had native heart failure and after drug therapy have either recovered or not their heart function. We discovered a proteomic biomarker panel of recovered heart function that not only worked in patients who recovered by means of transplantation but also in patients who recovered by means of drug therapy. The performance of these biomarkers is very clinically relevant thus will change heart failure patient management.

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

Terms used in the claims and specification are defined as set forth below unless otherwise specified.

“Marker” or “markers” or “biomarker,” “biomarkers,” refers generally to a molecule (typically nucleic acid, protein, carbohydrate, or lipid) that is expressed in cell or tissue, which is useful for the prediction of allograft rejection of heart transplants. In the case of a nucleic acid, a marker can include any allele, including wild-types alleles, SNPs, microsatellites, insertions, deletions, duplications, and translocations. A marker can also include a peptide encoded by a nucleic acid. A marker in the context of the present teachings encompasses, for example, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences.

To “analyze” includes measurement and/or detection of data associated with a marker (such as, e.g., presence or absence of a nucleic acid sequence, or protein, or constituent expression levels) in the sample (or, e.g., by obtaining a dataset reporting such measurements, as described below). In some aspects, an analysis can include comparing the measurement and/or detection of at least one marker in samples from a subject pre- and post-treatment or other control subject(s). The markers of the present teachings can be analyzed by any of various conventional methods known in the art.

A “subject” in the context of the present teachings is generally a mammal. The subject is generally a patient. The term “mammal” as used herein includes but is not limited to a human, non-human primate, dog, cat, mouse, rat, cow, horse, and pig. Mammals other than humans can be advantageously used as subjects that represent animal models of heart transplantation. A subject can be male or female.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

In particular aspects, the sample is a blood sample from the subject.

A “dataset” is a set of data (e.g., numerical values) resulting from evaluation of a sample. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, PCR, microarray, one or more primers, one or more probes, antibody binding, ELISA, or mass spectometry. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a marker or other substance (e.g., nucleic acid or protein) in a clinical or subject-derived sample, including the presence, absence, or concentration levels of such markers or substances, and/or evaluating the values or categorization of a subject's clinical parameters.

The term “expression level data” refers to a value that represents a direct, indirect, or comparative measurement of the level of expression of a polynucleotide (e.g., RNA or DNA) or polypeptide. For example, “expression data” can refer to a value that represents a direct, indirect, or comparative measurement of the protein expression level of a proteomic marker of interest.

### Markers and Clinical Factors

In an embodiment, the invention includes obtaining a first dataset associated with a sample obtained from the subject (e.g., a blood sample), wherein the first dataset comprises quantitative expression data for one or more mRNA or protein markers selected from Table 1. This first sample can be taken, for example, before treatment. In some embodiments, the invention further includes analyzing the first dataset to determine the expression level of the one or more mRNA or protein markers, wherein the expression level of the markers positively or negatively correlates with recovered heart function in a subject.

In another embodiment, the invention includes obtaining a second dataset associated with a sample obtained from the subject (e.g., another blood sample), wherein the second dataset comprises quantitative expression data for one or more mRNA or protein markers selected from Table 1. This second sample can be taken, for example, after treatment. In some embodiments, the invention further includes analyzing the second dataset to determine the expression level of the one or more mRNA or protein markers, wherein the expression level of the markers positively or negatively correlates with recovered heart function in a subject.

In additional embodiments, the analysis includes both the first dataset and second dataset, wherein the aggregate analysis of marker expression levels positively or negatively correlates with recovered heart function in a subject.

The quantity of one or more markers of the invention can be indicated as a value. A value can be one or more numerical values resulting from evaluation of a sample. The values can be obtained, for example, by experimentally obtaining measures from a sample by an assay performed in a laboratory, or alternatively, obtaining a dataset from a service provider such as a laboratory, or from a database or a server on which the dataset has been stored, e.g., on a storage memory.

In an embodiment, the quantity of one or more markers can be one or more numerical values associated with RNA or protein expression levels of probe sets and proteins shown in Table 1 below, e.g., resulting from evaluation of a patient derived sample.

A marker's associated value can be included in a dataset associated with a sample obtained from a subject. A dataset can include the marker expression value of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more marker(s). The value of the one or more markers can be evaluated by the same party that performed the assay using the methods of the invention or sent to a third party for evaluation using the methods of the invention.

In some embodiments, one or more clinical factors in a subject can be assessed. In some embodiments, assessment of one or more clinical factors or variables in a subject can be combined with a marker analysis in the subject to determine recovered heart function in a subject. Examples of relevant clinical factors or variables include, but are not limited to, left ventricular ejection fraction (LVEF) and New York Heart Association (NYHA) class.

### Assays

Examples of assays for one or more markers include sequencing assays, microarrays, polymerase chain reaction (PCR), RT-PCR, Southern blots, northern blots, antibody-binding assays, enzyme-linked immunosorbent assays (ELISAs), flow cytometry, protein assays, western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than assay information to a computer system, such as race, height, weight, age, sex, eye color, hair color, family medical history and any other information that may be useful to a user, such as a clinical factor or variable described herein.

### Nucleic Acids and Antibodies

Nucleic Acids, Portions and Variants

The nucleic acid molecules of the present invention can be RNA, for example, mRNA, or DNA, such as cDNA and genomic DNA. DNA molecules can be double-stranded or single-stranded; single-stranded RNA or DNA can be the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example).

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA/cDNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized.

An isolated nucleic acid molecule can include a nucleic acid molecule or nucleic acid sequence that is synthesized chemically or by recombinant means. Such isolated nucleic acid molecules are useful as probes for detecting expression of the gene in tissue (e.g., human tissue), such as using the methods disclosed herein.

Nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., markers). In one aspect, the invention includes variants described herein that hybridize under high stringency hybridization conditions (e.g., for selective hybridization) to a nucleotide sequence encoding an amino acid sequence or a polymorphic variant thereof.

Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity. “High stringency conditions,” “moderate stringency conditions” and “low stringency conditions,” as well as methods for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998)), and in Kraus, M. and Aaronson, S., Methods Enzymol., 200:546-556 (1991), incorporated herein, by reference.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain aspects, the length of a sequence aligned for comparison purposes is at least 30%, for example, at least 40%, in certain aspects at least 60%, and in other aspects at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., W=5 or W=20).

The present invention also provides isolated nucleic acid molecules that contain a fragment or portion that hybridizes under highly stringent conditions to a nucleotide sequence or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain aspects about 40, 50 or 75, consecutive nucleotides of a nucleic acid molecule comprising a contiguous nucleotide sequence or polymorphic variant thereof. In other aspects, a probe or primer comprises 100 or fewer nucleotides, in certain aspects from 6 to 50 nucleotides, for example from 12 to 30 nucleotides. In other aspects, the probe or primer is at least 70% identical to the contiguous nucleotide sequence or to the complement of the contiguous nucleotide sequence, for example at least 80% identical, in certain aspects at least 90% identical, and in other aspects at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention can be identified and isolated using standard molecular biology techniques and the sequence information provided herein. For example, nucleic acid molecules can be amplified and isolated by the polymerase chain reaction (PCR) using synthetic oligonucleotide primers designed based on the sequence of a nucleic acid sequence of interest or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleic acid sequences can be used as reagents in the screening and/or predictive assays described herein, and can also be included as components of kits (e.g., reagent kits) for use in the screening and/or predictive assays described herein.

Antibodies

Polyclonal antibodies and/or monoclonal antibodies that specifically bind to marker gene products are also provided. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. The invention provides polyclonal and monoclonal antibodies that bind to a polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well-known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. No. 4,946,778 and No. 5,260,203, the disclosures of which are incorporated by reference.

In general, antibodies of the invention (e.g., a monoclonal antibody) can be used to detect a polypeptide marker (e.g., in heart tissue or blood sample) in order to evaluate the abundance and pattern of expression of the polypeptide. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### Detection Assays

Nucleic acids, probes, primers, and antibodies such as those described herein can be used in a variety of methods to determine the expression levels of the markers disclosed herein, and thus, determine recovered heart function. In one aspect, kits can be made which comprise primers or antibodies that can be used to quantify the markers of interest.

In aspects of the invention, the determination of recovered heart function is made by determining the expression level of one or more markers of the invention. In one embodiment, a hybridization sample can be formed by contacting the test sample containing a nucleic acid with at least one nucleic acid probe. A probe for detecting mRNA cDNA can be a labeled nucleic acid probe capable of hybridizing to mRNA or cDNA sequences. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or cDNA.

The hybridization sample is maintained under conditions that are sufficient to allow specific hybridization of the nucleic acid probe to a nucleic acid. “Specific hybridization,” as used herein, indicates exact hybridization (e.g., with no mismatches). Specific hybridization can be performed under high stringency conditions or moderate stringency conditions, for example, as described above. In a particularly preferred aspect, the hybridization conditions for specific hybridization are high stringency.

In northern analysis (see Current Protocols in Molecular Biology, Ausubel, F. et al., eds., John Wiley & Sons.), a test sample of RNA is obtained from samples by appropriate means. Specific hybridization of a marker nucleic acid probe to mRNA from a sample can be quantitated to determine that marker's expression level.

Alternatively, a peptide nucleic acid (PNA) probe can be used instead of a nucleic acid probe in the hybridization methods. PNA is a DNA mimic having a peptide-like, inorganic backbone, such as N-(2-aminoethyl) glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a nucleic acid. Hybridization of the PNA probe to a nucleic acid can be used to determine a marker's expression level, and thus, serve to determine recovered heart function in a subject.

In another aspect, arrays of oligonucleotide probes that are complementary to target marker nucleic acid sequence segments from a sample can be used to quantitate the level of given markers. For example, in one aspect, an oligonucleotide array can be used. Oligonucleotide arrays typically comprise a plurality of different oligonucleotide probes that are coupled to a surface of a substrate in different known locations. These oligonucleotide arrays have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of which are incorporated by reference herein. Techniques for the synthesis of these arrays using mechanical synthesis methods are described in, e.g., U.S. Pat. No. 5,384,261; the entire teachings are incorporated by reference herein. In another example, linear arrays can be utilized.

Once an oligonucleotide array is prepared, a nucleic acid of interest is hybridized with the array and scanned for levels of hybridization. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein.

In one aspect of the invention, expression analysis by quantitative RT-PCR may also be used. These techniques, utilizing, e.g., TaqMan assays or DNA binding dyes, such as SYBR-GREEN, can assess the levels of expression of the markers of the invention.

In another aspect of the invention, expression levels of polypeptide markers can be measured using a variety of methods, including enzyme linked immunosorbent assays (ELISAs), western blots, immunoprecipitations and immunofluorescence. A test sample from a subject is subjected a measurement of protein expression levels using marker-specific antibodies.

Various means of examining expression or composition of the polypeptide encoded by a nucleic acid can be used, including: spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly Chapter 10). For example, in one aspect, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling a DNA probe with biotin such that it can be detected with fluorescently labeled streptavidin.

### Computer Implementation

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

### EXAMPLES

Below are examples of specific embodiments of the invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of embodiments of the invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

The goal of our work discussed below was to identify biomarkers useful for determining recovered heart function in a subject.

### Example 1

### General Materials and Methods and Study Cohorts

### Patient Cohorts

All patients included in the study were enrolled as part of the BiT and Validation of Cured Heart Failure initiatives approved by the Providence Health Care Research Ethics Board. Patients were approached by the clinical coordinators and those who gave informed consent were enrolled in the study.

### Discovery Cohort

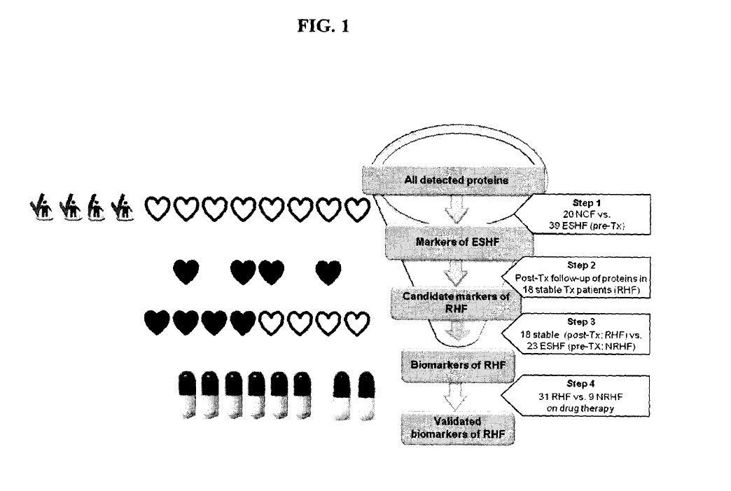

To facilitate the identification of biomarkers of recovered heart function heart transplant patients enrolled as part of the BiT initiative were included in the discovery analysis. This cohort was ideal since blood samples were collected from enrolled patients on average within two weeks prior to transplantation as well as longitudinally post-transplant. A total of 41 transplant patients' pre- and/or post-transplant samples were included in the analysis. Twenty non-transplant individuals with normal cardiac function (NCF) were also selected for proteomic analysis. In order to study heart failure markers, 39 patients' pre-transplant samples (end-stage heart failure; ESHF) and 20 NCF were selected for statistical analysis (FIG. 1). To identify biomarkers of recovered heart function, the analysis focused on non-rejection samples collected between weeks two and four and year one post-transplant from 18 patients deemed stable by the clinical team (FIG. 1, step 2). Stable transplant patients had no treatable acute rejection episodes, no right sided heart failure, no kidney dysfunction, no anemia, and did not have infections that required antibiotics treatment within one month or at year one post-transplant.

### Validation Cohort

The biomarkers discovered in the first phase of the study were validated in 40 patients who had heart failure for at least one year and were treated with standard HF drug therapy and either recovered or have not. The 31 patients who recovered their heart function using drug therapy were enrolled from the Maintenance Clinic at St. Paul's Hospital, Vancouver, Canada, which provides specialized care to patients with heart failure. All 31 patients had left ventricular ejection fraction (LVEF) of 50% or higher and New York Heart Association (NYHA) class I. These patients had an improvement in LVEF of at least 25% since HF diagnosis. There were 9 patients whose heart function did not improve after at least one year of drug therapy. These patients were enrolled either from the Heart Failure Clinic or the inpatient ward at St. Paul's Hospital and had LVEF of 25% or less and NYHA class III or IV.

### Sample Collection, Processing in Proteomic Analysis

Blood samples were collected in EDTA tubes (BD, Franklin Lake, N.J., USA) and stored on ice until processing. Blood was spun down within two hours of collection and plasma was stored at −80° C. until selected for proteomic analysis.

### Discovery Platform

The discovery cohort samples were analyzed using iTRAQ proteomics as previously described. Briefly, one aliquot of plasma was depleted of the 14 most abundant proteins, according to Standard Operating Procedure, and sent to the UVic Genome BC Proteomics Centre, Victoria, Canada, for proteomic analysis. Identification and quantitation of peptides and proteins was determined by iTRAQ labelling and 2D-LC-MS/MS on ABI 4800 Mass Spectrometers. The raw data was analyzed using ProteinPilot™ 3.0 software (Applied Biosystem) and with International Protein Index (IPI) database v3.67. The protein levels for the patient samples, reported by ProteinPilot™, were relative to a pool of samples collected from 16 healthy individuals. PGCs were assembled based on ProteinPilot™ output and an in-house algorithm called Protein Group Code Algorithm.

### Validation Platform

The validation cohort's plasma samples were analyzed using Mass Spectrometry based Multiple Reaction Monitoring (MRM). MRM have been used for detecting small molecules but only recently started to be employed as a validation quantitative proteomics platform. For this study, MRM assays were developed for all protein groups in the discovered biomarker panel, based on one or two unique peptides per protein group. The peptide levels, corresponding to the proteins in the RHF biomarker panel, were quantified in one multiplex run. The peptides were also measured in a pool of 16 healthy individuals, the same as the one used in the iTRAQ analysis of the discovery cohort samples. In order to make the MRM data comparable with the iTRAQ data, relative peptide levels were calculated by dividing each patient's data by the pooled normal.

### Statistical Analysis

The statistical analysis of the data was performed using R (www.r-project.org) and Bioconductor (www.bioconductor.org).

### Biomarker Discovery

The biomarker discovery was performed within the transplant model by applying the analysis pipeline outlined in FIG. 1. In step one, protein markers of HF were identified. Proteins that were only present in less than 25% of the discovery cohort patient samples were eliminated. The proteomics data was log 2 transformed. Thirty-nine ESHF and 20 NCF samples were compared by means of a moderated t-test developed for the analysis of omics data called limma. False Discovery Rate (FDR) of less than 0.05 was considered statistically significant. In step two, the level of the proteins identified in the previous step was followed over time post-transplant, and those with levels reverting to normal by the first month and staying within the normal range at one year post-transplant were identified. The normal range was calculated separately for each protein based on mean±two standard deviation of the level of NCF samples. In step three, the candidate biomarkers of RHF were compared by means of limma between week two to four samples of stable patients (RHF group) and ESHF, pre-transplant samples of independent patients (NRHF group). Those with FDR <0.1 were considered biomarkers of RHF. The biomarker panel protein groups were analyzed with elastic net classification method which built a classifier using these proteins.

### Biomarker Validation

The level of each protein group in the RHF biomarker panel were compared one at a time in the validation RHF versus NRHF patients by means of Student's t-test. The Bioconductor package globaltest was used to test if the global expression pattern of all the proteins in the biomarker panel are also associated with heart function status. In addition, confounder analyses were performed using globaltest in order to assess if any of the medications are also associated with the global expression of the biomarker protein groups. All drug and/or drug types given to at least two validation cohort patients were included in the confounder analysis: digoxin, aspirin, warfarin, amiodarone, beta blockers, angiotensin converting enzyme (ACE) inhibitors, angiotensin receptor blockers (ARB), statins, diuretics, and anti arrhythmia drugs. For the drugs with p-value <0.05, globaltest was applied to verify if heart failure recovery status was significant independent of the specific drug.

### Example 2

### Biomarker Discovery

The biomarkers of RHF were discovered in several analytical steps, as described in FIG. 1. This involved first identifying markers of ESHF in the pre-transplant patient samples and following these markers in the longitudinally collected post-transplant samples of stable patients to see which ones normalize and stay within normal levels after a year post-transplant.

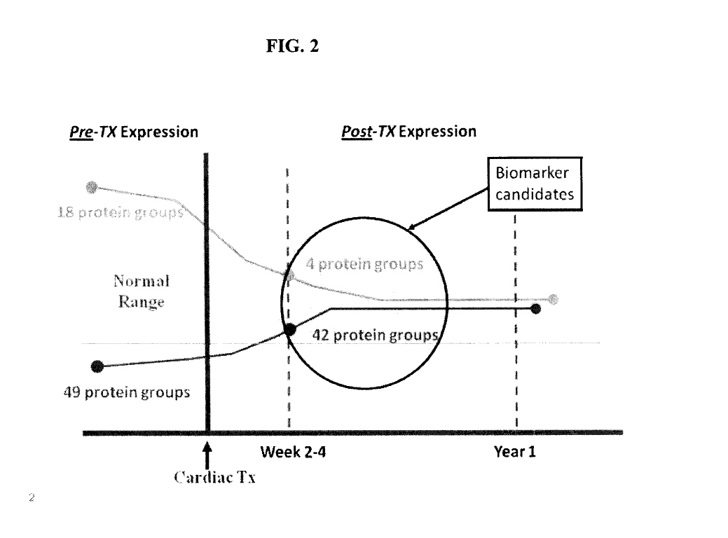

The proteomic data of 20 individuals with normal cardiac function were compared to 39 heart transplant patients' pre-transplant, ESHF, samples. The analysis revealed that 67 protein groups were differentially abundant in ESHF relative to NCF samples, had a FDR <0.05 (FIG. 2), indicating that these 67 protein groups are markers of ESHF. Of these, 18 were observed at higher levels of abundance in ESHF relative to NCF samples.

Of the 67 protein markers of ESHF, 46 reversed back to normal levels by month 1 and also stayed normal at year 1 (FIG. 2). These 46 were considered candidate proteomic markers of RHF and were compared between RHF (post-transplant patient samples) and NRHF (pre-transplant patient samples independent of the RHF patients). A total of 18 had differential levels between these groups with FDR <0.1. The final biomarker panel was built by means of elastic net using the relative to pooled normal levels of these 18 protein groups.

### Example 3

### Biomarker Validation

MRM assay was developed for 1 or 2 peptides that were unique to each of the 18 protein groups in the biomarker panel. For the protein groups with 2 peptides the level of the protein was calculated by using the peptide with the highest level in most of the validation cohort samples. The peptide AYSLFSYNTQGR, corresponding to serum-amyloid P component precursor, was not detected in any of the samples. Since this was the only peptide selected for this protein, the biomarker panel was recalibrated, i.e. the classifier was re-built, in the discovery cohort using information from the other 17 protein groups only. Thus, the final biomarker of RHF contained 17 proteins.

The p-value was calculated for each of the 17 protein groups. Based on Student's t-test, 12 of them were statistically significant, had p-value <0.05. In the next step, the global expression of the protein groups was tested because they would be used together in clinical decision making. The p-value corresponding to the global expression of the 17 protein groups was 0.00006.

In order to assess if the discovered biomarker panel was truly associated with recovered heart function and not medication, analyses were performed for each drug given to the validation patients. The confounder analyses of the drug therapy revealed that warfarin, ARBs and diuretics had p-value <0.05 indicating that they were associated with the biomarker proteins. The globaltest applied to inquire if heart failure recovery status was significant independent of the warfarin, ARBs and diuretics resulted in p-values of 0.0003, 0.0005 and 0.0054 respectively, indicating that the global expression of the 17 protein groups was indicative of RHF independent of the medications taken by the patients.

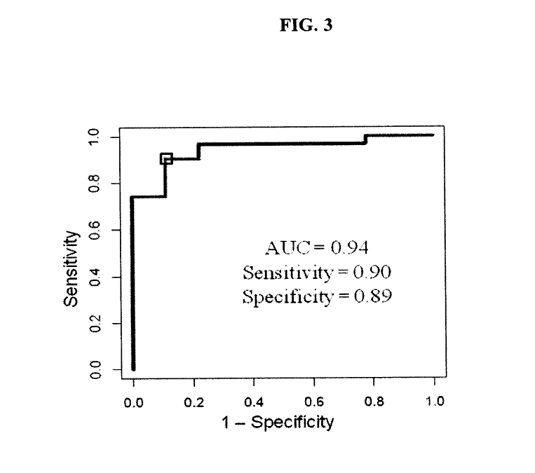

As a last validation step, the biomarker score was calculated for each validation patient by applying the 17 protein group based classifier. Based on the scores, the biomarker's AUC=0.94 (FIG. 3). At the point on the ROC curve marked with the blue square (FIG. 3), the sensitivity and specificity were 0.90 and 0.89, respectively.

The biomarkers can be ordered using the following scheme. The initial ordering can be based on the weights assigned by the elastic net classification method. These weights are assigned when the model is built in the discovery cohort. P-values obtained based on the Students t-test applied to the validation cohort are considered in the final ranking. Thus, the proteins with very small weights or large p-values are placed at the bottom of the list.

### DISCUSSION

Although some patients appear to recover from the symptomatic phase of heart failure, their treatment is continued. This is due to the fact that currently there are no guidelines for assessing whether a patient has been “cured” of heart failure. Without proper guidelines, clinicians are obligated to continue the standard heart failure treatment in order to avoid a relapse from deteriorating cardiac function. Biomarkers of RHF would help physicians tailor the treatment decision to each individual patient, which could save costs and reduce side effects and complications over time.

In this study, a unique approach was taken for discovering biomarkers of RHF by testing the hypothesis that biomarkers of cured heart failure are equivalent in patients with stable heart function managed medically versus those with cardiac transplant. In the heart transplant setting, patients have end-stage heart failure before transplantation and after receiving the new heart, they may be cured of heart failure by their new graft. This post-transplant salvage of heart function served as an excellent model for studying proteins indicative of recovering heart.

### Clinical Use

Since the biomarker panel provides a determination of recovered heart function, it can be used to improve the management of care for patients who have suffered and are recovering from heart failure. Among these benefits include: Patients could be tested locally instead of needing to travel to a tertiary care center. Patients with RHF could be followed less frequently. Patients with RHF could be weaned off of medication, depending on original cause of 1-1F. This would result in fewer complications due to drug side effects. Patients with NRHF could be potentially followed more often and provided the proper medical treatments at earlier stages.

### REFERENCES

1. Chen et al., National and Regional Trends in Heart Failure Hospitalization and Mortality Rates for Medicare Beneficiaries, 1998-2008, JAMA 2011—Vol 30
2. J. Paul Rocchiccioli et al., Biomarkers in heart failure: a clinical review, Heart failure Rev., 2010
3. Frank Kramer et al., Novel biomarkers in human terminal heart failure and under mechanical circulatory support, Biomarkers, 2011
4. Ariadne Avellino et al., Risk stratification and short-term prognosis in acute heart failure syndromes: A review of novel biomarkers, Biomarkers, 2011
5. Leanne E. Felkin et al., Expression of Extracellular Matrix Genes During Myocardial Recovery From Heart Failure After Left Ventricular Assist Device Support, J Heart Lung Transplant 2009
6. Shamim Ahmad et al., Circulating proinflammatory cytokines and N-terminal pro-brain natriuretic peptide significantly decrease with recovery of left ventricular function in patients with dilated cardiomyopathy, Mol Cell Biochem (2009)
7. Roger V L, Weston S A, Redfield M M, et al. Trends in heart failure incidence and survival in a community-based population. JAMA. 2004; 292(3):344:50.

8. Smyth G. Limma: linear models for microarray data, in Bioinformatics and Computational Biology Solutions using R and Bioconductor, R. Gentleman, et al., Editors. 2005, Springer: New York.

9. Kuzyk M A, et al. Multiple reaction monitoring-based, multiplexed, absolute quantitation of 45 proteins in human plasma. Mol Cell Proteomics 2009; 8: 1860-77
10. Zou H, Hastie T. Regularization and variable selection via the elastic net. J R Stat Soc B Stat Methodol 2005; 67: 301.
11. Goeman, J J, et al. A global test for groups of genes: testing association with a clinical outcome. Bioinformatics 2004; 20: 93-9.

TABLE 1
RHF biomarker panel proteins

| | | | | In Final |
|---|---|---|---|---|
| Protein Name Detected in iTRAQ | Gene | | Protein Detected in MRM | Biomarker |
| Discovery Cohort | Symbol | MRM Assay Peptide | Validation Cohort | Panel |
| cDNA FLJ58075, highly similar to | CP | GAYPLSIEPIGVR* | Ceruloplasmin (ferroxidase) | Yes |
| Ceruloplasmin | | IYHSHIDAPK | | |
| Ceruloplasmin | | | | |
| cDNA FLJ37971 fis, clone | | | | |
| CTONG2009958, highly similar to | | | | |
| CERULOPLASMIN | | | | |
| Putative uncharacterized protein | | | | |
| CP | | | | |
| Inter-alpha (Globulin) inhibitor | ITIH2 | ETAVDGELVVLYDVK* | Inter-alpha-trypsin inhibitor | Yes |
| H2 | | FLHVPDTFEGHFDGVPVIS | heavy chain H2 | |
| Inter-alpha-trypsin inhibitor | | K | | |
| heavy chain H2 | | | | |
| Angiotensinogen | AGT | ALQDQLVLVAAK | Angiotensinogen | Yes |
| Antithrombin-III | SERPINC1 | DDLYVSDAFHK | Antithrombin-III | Yes |
| SERPINC1 protein | | | | |
| Prothrombin (Fragment) | F2 | ETAASLLQAGYK | Prothrombin | Yes |
| insulin-like growth factor binding | IGFALS | VAGLLEDTFPGLLGLR* | insulin-like growth factor | Yes |
| protein, acid labile subunit | | NLIAAVAPGAFLGLK | binding protein, acid labile | |
| isoform 1 precursor | | | subunit | |
| Insulin-like growth factor-binding | | | | |
| protein complex acid labile chain | | | | |
| Alpha-2-antiplasmin | SERPINF2 | LGNQEPGGQTALK | Alpha-2-antiplasmin | Yes |
| alpha-2-antiplasmin isoform b | | | | |
| precursor | | | | |
| Putative uncharacterized protein | | | | |
| SERPINF2 | | | | |
| 55 kDa protein | — | | | |
| Apolipoprotein A-I | APOA1 | ATEHLSTLSEK | Apolipoprotein A-I | Yes |
| Apolipoprotein A1 | | | | |
| CLU | CLU | ELDESLQVAER | Clusterin | Yes |
| Isoform 2 of Clusterin | | | | |
| Isoform 1 of Clusterin | | | | |
| 54 kDa protein | | | | |
| Vitronectin | VTN | FEDGVLDPDYPR | Vitronectin | Yes |
| C4b-binding protein alpha chain | C4BPA | EDVYVVGTVLR* | C4b-binding protein alpha | Yes |
| Putative uncharacterized protein | | LSLEIEQLELQR | chain | |
| C4BPA | | | | |
| Serum amyloid P-component | APCS | AYSLFSYNTQGR** | Serum-amyloid P component | No |
| | | | precursor | |
| Vitamin K-dependent protein S | PROS1 | VYFAGFPR* | Vitamin K-dependent protein S | Yes |
| PROS1 protein | | SFQTGLFTAAR | Vitamin K-dependent protein S | |
| cDNA FLJ56936, highly similar to | | TSLGSDSSTQAK** | Adenylate cyclase type 9 | |
| Vitamin K-dependent protein S | | TLDEILQEK** | cDNA FLJ55257, moderately | |
| Adenylate cyclase type 9 | ADCY9 | TSNLLLSHAGILK** | similar to PITSLRE | |
| Isoform SV2 of PITSLRE | CDC2L2 | | serine/threonine-protein | |
| serine/threonine-protein kinase | | | kinaseCDC2L2 | |
| CDC2L2 | | | Isoform SV2 of PITSLRE | |
| cDNA FU55257, moderately | — | | serine/threonine-protein | |
| similar to PITSLRE | | | kinase CDC2L2 | |
| serine/threonine-protein | | | | |
| kinaseCDC2L2 | | | | |
| RB1-inducible coiled-coil protein | RB1CC1 | | | |
| 1 | | | | |
| Rb1-inducible coiled coil protein | | | | |
| 1 isoform 2 | | | | |
| Coagulation factor X | F10 | TGIVSGFGR* | Coagulation factor X | Yes |
| Coagulation factor X | | ETYDFDIAVLR | | |
| Putative uncharacterized protein | | | | |
| F10 | | | | |
| Vitamin K-dependent protein C | PROC | YLDWIHGHIR* | Vitamin K-dependent | Yes |
| Protein C (Fragment) | | TFVLNFIK | protein C | |
| Putative uncharacterized protein | | | | |
| PROC | | | | |
| cDNA FLJ51034, highly similar to | | | | |
| Vitamin K-dependent protein C | | | | |
| cDNA FLJ51925, highly similar to | | | | |
| Vitamin K-dependent protein C | | | | |
| cDNA FLJ51179, highly similar to | | | | |
| Vitamin K-dependent protein C | | | | |
| Isoform A of Proteoglycan 4 | PRG4 | GFGGLTGQIVAALSTAK* | Proteoglycan 4 | Yes |
| Isoform B of Proteoglycan 4 | | DQYYNIDVPSR** | | |
| Isoform C of Proteoglycan 4 | | | | |
| Isoform D of Proteoglycan 4 | | | | |
| Isoform E of Proteoglycan 4 | | | | |
| Isoform F of Proteoglycan 4 | | | | |
| Coagulation factor IX | F9 | VSVSQTSK* | Coagulation factor IX | Yes |
| Coagulation factor IX (Fragment) | | SALVLQYLR | | |
| Isoform 4 of E3 ubiquitin-protein | UBR4 | | | |
| ligase UBR4 | | | | |
| Isoform 1 of E3 ubiquitin-protein | | | | |
| ligase UBR4 | | | | |
| Isoform 5 of E3 ubiquitin-protein | | | | |
| ligase UBR4 | | | | |
| Isoform 3 of E3 ubiquitin-protein | | | | |
| ligase UBR4 | | | | |
| Isoform 2 of E3 ubiquitin-protein | | | | |
| ligase UBR4 | | | | |
| Complement factor D | CFD | THHDGAITER | Complement factor D | Yes |
| preproprotein | | | | |

*= peptide with highest levels
**= not detected in MRM

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1.-3. (canceled)

4. A method for determining recovered heart function a subject, comprising:
obtaining a first dataset associated with a first sample obtained from a subject before treatment, wherein the first dataset comprises at least one marker selected from Table 1;
obtaining a second dataset associated with a second sample obtained from the subject after treatment, wherein the second dataset comprises at least one marker selected from Table 1;
analyzing the first and second datasets to determine data for the markers, wherein the data is positively correlated or negatively correlated with recovered heart function in the subject.

5. The method of claim 4, wherein the first dataset comprises data for at least two, three, four, five, six, seven, eight, nine, ten, eleven, twelve, thirteen, fourteen, fifteen, sixteen, seventeen, eighteen, nineteen, twenty or more markers selected from Table 1,
and wherein the second dataset comprises data for at least two, three, four, five, six, seven, eight, nine, ten, eleven, twelve, thirteen, fourteen, fifteen, sixteen, seventeen, eighteen, nineteen, twenty or more markers selected from Table 1;
and further comprising analyzing the first and second datasets to determine the expression level of the at least two, three, four, five, six, seven, eight, nine, ten, eleven, twelve, thirteen, fourteen, fifteen, sixteen, seventeen, eighteen, nineteen, twenty or more markers selected from Table 1.

6. The method of claim 5, further comprising determining recovered heart function in the subject according to the relative number of positively correlated and negatively correlated marker expression level data present in the first and second datasets.

7. The method of claim 4, wherein the sample obtained from the subject is a blood sample.

8. The method of claim 4, wherein the data is protein expression data.

9. The method of claim 8, wherein the protein expression data is obtained using mass spectrometry.

10. The method of claim 8, wherein the protein expression data is obtained using an antibody.

11. The method of claim 10, wherein the antibody is labeled.

12. The method of claim 4, wherein the method is implemented using one or more computers.

13. The method of claim 4, wherein the first and/or second dataset is stored on a storage memory.

14. The method of claim 4, wherein obtaining the first and/or second dataset comprises receiving the first and/or second dataset directly or indirectly from a third party that has processed the sample to experimentally determine the first and/or second dataset.

15. The method of claim 4, wherein the subject is a human subject.

16.-17. (canceled)

18. A method for determining recovered heart function in a subject, comprising:
obtaining a first sample from the subject before treatment, wherein the first sample comprises at least one marker selected from Table 1;
obtaining a second sample from the subject after treatment, wherein the second sample comprises at least one marker selected from Table 1;
contacting the first and second samples with a reagent;
generating a complex between the reagent and the markers;
detecting the complex to obtain a dataset associated with the samples, wherein the dataset comprises expression level data for the markers; and
analyzing the expression level data for the markers, wherein the expression level of the markers is positively correlated or negatively correlated with recovered heart function in the subject.

19.-21. (canceled)

22. A system for determining recovered heart function in a subject, the system comprising:
a storage memory for storing a first dataset associated with a first sample obtained from the subject before treatment, wherein the first dataset comprises data for at least one marker selected from Table 1;
a storage memory for storing a second dataset associated with a second sample obtained from the subject after treatment, wherein the second dataset comprises data for at least one marker selected from Table 1; and
a processor communicatively coupled to the storage memory for analyzing the first and second datasets to determine the expression levels of the markers, wherein the expression levels are positively correlated or negatively correlated with recovered heart function in the subject.

23.-27. (canceled)

28. The method of claim 4, wherein the treatment is administration of a beta-blocker or ACE inhibitor.

29. The system of claim 22, wherein the treatment is administration of a beta-blocker or ACE inhibitor.

30. The method of claim 4, wherein the at least one marker selected from Table 1 is selected from the group consisting of: Ceruloplasmin (ferroxidase) (CP); Alpha-2-antiplasmin (SERPINF2); Prothrombin (F2); Proteoglycan 4 (PRG4); Inter-alpha-trypsin inhibitor heavy chain H2 (ITIH2); Vitamin K-dependent protein S (PROS1); complement factor D (CFD); Coagulation factor X (F10); Vitamin K-dependent protein C (PROC); Apolipoprotein A-I (APOA1); Clusterin (CLU); C4b-binding protein alpha chain (C4BPA); Vitronectin (VTN); Antithrombin-III (SERPINC1); coagulation factor IX (F9); insulin-like growth factor binding protein, acid labile subunit (IGFALS); angiotensinogen (AGT); and Serum amyloid P-component (APCS).

31. The method of claim 30, wherein the at least one marker is Ceruloplasmin (ferroxidase) (CP).

32. The method of claim 18, wherein the at least one marker selected from Table 1 is selected from the group consisting of: Ceruloplasmin (ferroxidase) (CP); Alpha-2-antiplasmin (SERPINF2); Prothrombin (F2); Proteoglycan 4 (PRG4); Inter-alpha-trypsin inhibitor heavy chain H2 (ITIH2); Vitamin K-dependent protein S (PROS1); complement factor D (CFD); Coagulation factor X (F10); Vitamin K-dependent protein C (PROC); Apolipoprotein A-I (APOA1); Clusterin (CLU); C4b-binding protein alpha chain (C4BPA); Vitronectin (VTN); Antithrombin-III (SERPINC1); coagulation factor IX (F9); insulin-like growth factor binding protein, acid labile subunit (IGFALS); angiotensinogen (AGT); and Serum amyloid P-component (APCS).

33. The method of claim 32, wherein the at least one marker is Ceruloplasmin (ferroxidase) (CP).


SEQUENCE LISTING (30 sequences)

SEQ ID NO: 1 - protein, 13 aa, Artificial Sequence
  synthetic recovered heart function (RHF) biomarker Mass Spectrometry based Multiple Reaction Monitoring (MRM) assay peptide ceruloplasmin (ferroxidase) (CP)
GAYPLSIEPIGVR

SEQ ID NO: 2 - protein, 10 aa, Artificial Sequence
  synthetic recovered heart function (RHF) biomarker Mass Spectrometry based Multiple Reaction Monitoring (MRM) assay peptide ceruloplasmin (ferroxidase) (CP)
IYHSHIDAPK

SEQ ID NO: 3 - protein, 15 aa, Artificial Sequence
  synthetic recovered heart function (RHF) biomarker Mass Spectrometry based Multiple Reaction Monitoring (MRM) assay peptide inter-alpha-trypsin inhibitor heavy chain H2 (ITIH2)
ETAVDGELVVLYDVK

SEQ ID NO: 4 - protein, 20 aa, Artificial Sequence
  synthetic recovered heart function (RHF) biomarker Mass Spectrometry based Multiple Reaction Monitoring (MRM) assay peptide inter-alpha-trypsin inhibitor heavy chain H2 (ITIH2)
FLHVPDTFEGHFDGVPVISK

SEQ ID NO: 5 - protein, 12 aa, Artificial Sequence
  synthetic recovered heart function (RHF) biomarker Mass Spectrometry based Multiple Reaction Monitoring (MRM) assay peptide angiotensinogen (AGT)
ALQDQLVLVAAK

SEQ ID NO: 6 - protein, 11 aa, Artificial Sequence
  synthetic recovered heart function (RHF) biomarker Mass Spectrometry based Multiple Reaction Monitoring (MRM) assay peptide antithrombin-III (SERPINC1)
DDLYVSDAFHK

SEQ ID NO: 7 - protein, 12 aa, Artificial Sequence
  synthetic recovered heart function (RHF) biomarker Mass Spectrometry based Multiple Reaction Monitoring (MRM) assay peptide prothrombin (F2)
ETAASLLQAGYK

SEQ ID NO: 8 - protein, 16 aa, Artificial Sequence
  synthetic recovered heart function (RHF) biomarker Mass Spectrometry based Multiple Reaction Monitoring (MRM) assay peptide insulin-like growth factor binding protein, acid labile subunit (IGFALS)
VAGLLEDTFPGLLGLR

SEQ ID NO: 9 - protein, 15 aa, Artificial Sequence
  synthetic recovered heart function (RHF) biomarker Mass Spectrometry based Multiple Reaction Monitoring (MRM) assay peptide insulin-like growth factor binding protein, acid labile subunit (IGFALS)
NLIAAVAPGAFLGLK

SEQ ID NO: 10 - protein, 13 aa, Artificial Sequence
  synthetic recovered heart function (RHF) biomarker Mass Spectrometry based Multiple Reaction Monitoring (MRM) assay peptide alpha-2-antiplasmin (SERPINF2)
LGNQEPGGQTALK

SEQ ID NO: 11 - protein, 11 aa, Artificial Sequence
  synthetic recovered heart function (RHF) biomarker Mass Spectrometry based Multiple Reaction Monitoring (MRM) assay peptide apolipoprotein A-1 (APOA1)
ATEHLSTLSEK

SEQ ID NO: 12 - protein, 11 aa, Artificial Sequence
  synthetic recovered heart function (RHF) biomarker Mass Spectrometry based Multiple Reaction Monitoring (MRM) assay peptide clusterin (CLU)
ELDESLQVAER

SEQ ID NO: 13 - protein, 12 aa, Artificial Sequence
  synthetic recovered heart function (RHF) biomarker Mass Spectrometry based Multiple Reaction Monitoring (MRM) assay peptide vitronectin (VTN)
FEDGVLDPDYPR

SEQ ID NO: 14 - protein, 11 aa, Artificial Sequence
  synthetic recovered heart function (RHF) biomarker Mass Spectrometry based Multiple Reaction Monitoring (MRM) assay peptide C4b-binding protein alpha chain (C4BPA)
EDVYVVGTVLR

SEQ ID NO: 15 - protein, 12 aa, Artificial Sequence
  synthetic recovered heart function (RHF) biomarker Mass Spectrometry based Multiple Reaction Monitoring (MRM) assay peptide C4b-binding protein alpha chain (C4BPA)
LSLEIEQLELQR

SEQ ID NO: 16 - protein, 12 aa, Artificial Sequence
  synthetic recovered heart function (RHF) biomarker Mass Spectrometry based Multiple Reaction Monitoring (MRM) assay peptide serum-amyloid P component precursor (APCS)
AYSLFSYNTQGR

SEQ ID NO: 17 - protein, 8 aa, Artificial Sequence
  synthetic recovered heart function (RHF) biomarker Mass Spectrometry based Multiple Reaction Monitoring (MRM) assay peptide vitamin K-dependent protein S (PROS1)
VYFAGFPR

SEQ ID NO: 18 - protein, 11 aa, Artificial Sequence
  synthetic recovered heart function (RHF) biomarker Mass Spectrometry based Multiple Reaction Monitoring (MRM) assay peptide vitamin K-dependent protein S (PROS1)
SFQTGLFTAAR

SEQ ID NO: 19 - protein, 12 aa, Artificial Sequence
  synthetic recovered heart function (RHF) biomarker Mass Spectrometry based Multiple Reaction Monitoring (MRM) assay peptide vitamin K-dependent protein S (PROS1)
TSLGSDSSTQAK

SEQ ID NO: 20 - protein, 9 aa, Artificial Sequence
  synthetic recovered heart function (RHF) biomarker Mass Spectrometry based Multiple Reaction Monitoring (MRM) assay peptide vitamin K-dependent protein S (PROS1)
TLDEILQEK

SEQ ID NO: 21 - protein, 13 aa, Artificial Sequence
  synthetic recovered heart function (RHF) biomarker Mass Spectrometry based Multiple Reaction Monitoring (MRM) assay peptide vitamin K-dependent protein S (PROS1)
TSNLLLSHAGILK

SEQ ID NO: 22 - protein, 9 aa, Artificial Sequence
  synthetic recovered heart function (RHF) biomarker Mass Spectrometry based Multiple Reaction Monitoring (MRM) assay peptide coagulation factor X (F10)
TGIVSGFGR

SEQ ID NO: 23 - protein, 11 aa, Artificial Sequence
  synthetic recovered heart function (RHF) biomarker Mass Spectrometry based Multiple Reaction Monitoring (MRM) assay peptide coagulation factor X (F10)
ETYDFDIAVLR

SEQ ID NO: 24 - protein, 10 aa, Artificial Sequence
  synthetic recovered heart function (RHF) biomarker Mass Spectrometry based Multiple Reaction Monitoring (MRM) assay peptide vitamin K-dependent protein C (PROC)
YLDWIHGHIR

SEQ ID NO: 25 - protein, 8 aa, Artificial Sequence
  synthetic recovered heart function (RHF) biomarker Mass Spectrometry based Multiple Reaction Monitoring (MRM) assay peptide vitamin K-dependent protein C (PROC)
TFVLNFIK

SEQ ID NO: 26 - protein, 17 aa, Artificial Sequence
  synthetic recovered heart function (RHF) biomarker Mass Spectrometry based Multiple Reaction Monitoring (MRM) assay peptide proteoglycan 4 (PRG4)
GFGGLTGQIVAALSTAK

SEQ ID NO: 27 - protein, 11 aa, Artificial Sequence
  synthetic recovered heart function (RHF) biomarker Mass Spectrometry based Multiple Reaction Monitoring (MRM) assay peptide proteoglycan 4 (PRG4)
DQYYNIDVPSR

SEQ ID NO: 28 - protein, 8 aa, Artificial Sequence
  synthetic recovered heart function (RHF) biomarker Mass Spectrometry based Multiple Reaction Monitoring (MRM) assay peptide coagulation factor IX (F9)
VSVSQTSK

SEQ ID NO: 29 - protein, 9 aa, Artificial Sequence
  synthetic recovered heart function (RHF) biomarker Mass Spectrometry based Multiple Reaction Monitoring (MRM) assay peptide coagulation factor IX (F9)
SALVLQYLR

SEQ ID NO: 30 - protein, 10 aa, Artificial Sequence
  synthetic recovered heart function (RHF) biomarker Mass Spectrometry based Multiple Reaction Monitoring (MRM) assay peptide complement factor D preproprotein (CFD)
THHDGAITER